(19) United States
(12) Patent Application Publication
(10) Pub. No.: US 2004/0037889 A1
(43) Pub. Date: Feb. 26, 2004

(54) Stabilized, dry pharmaceutical compositions for drug delivery and methods of preparing same

(76) Inventors: Rodger J. Richeal, Campbell, CA (US); Paul R. Hemmes, San Jose, CA (US); George C. Y. Chiou, College Station, TX (US)

Correspondence Address: Charles D. Holland, Morrison & Foerster LLP, 755 Page Mill Road, Palo Alto, CA, 94304-1018, US

(21) Appl. No.: 10/301,765

(22) Filed: Nov. 20, 2002

Related U.S. Application Data
(63) Continuation of application No. 09/870,343, filed on May 29, 2001, now abandoned.
Application No. 09/870,343 is a continuation of application No. 08/965,660, filed on Nov. 6, 1997, now abandoned.

(51) Int. Cl. A61K 9/16; A61K 9/50; B01J 13/02; B01J 13/04

(52) U.S. Cl. 424/490; 264/4.1

(57) ABSTRACT

Dry, stabilized pharmaceutical spheres comprising a precisely measured amount of the pharmaceutical and a filler material that facilitates the immediate dissolution of the pharmaceutical upon contact with a solution are provided as well as methods for preparing same.

### TECHNICAL FIELD OF THE INVENTION

This invention relates to novel methods of producing a stable, dry formulation of pharmaceuticals that can be reconstituted to a liquid solution of precisely known concentration. In particular it relates to methods useful in stabilizing and then reconstituting drug solutions for application in the form or eye drops.

### BACKGROUND OF THE INVENTION

Solutions of pharmaceuticals must be precisely formulated in order to avoid both overdoses and inadequate treatment. In addition, the drug must be stable over time to avoid two problems, reduction in the potency of active ingredients due to decomposition, and the possible side effects of the decomposition products of the drug. One well known approach to solving this problem is to provide a drug in a dry form such as a tablet or capsule, or in a solid form such as a powder or lyophilized mass that can be mixed with water or other appropriate solvent and reconstituted.

Therapeutic drugs have traditionally been administered orally or by injection. However, a number of pharmaceuticals are not easily administered via these methods. For example, many drugs, particularly peptides, are degraded by digestive enzymes and/or the acidity present in the gastrointestinal tract and cannot be taken orally. Additionally, many substances are not readily absorbed in the gastrointestinal tract due to the low permeability of the intestinal membrane to hydrophilic compounds. Thus, these drugs must be administered parenterally.

An alternative method drug delivery is the direct injection of a drug solution into the blood stream, intravenous administration. This method, however, is generally painful, must be administered under sterile conditions to prevent the spread of infectious diseases, and precautions must be taken to avoid other potential problems caused by improperly administered injections and to insure safe handling of contaminated syringes and needles. Additionally, repeated injections, often necessary to control such chronic diseases such as diabetes mellitus, can cause undesirable side effects such as necrosis, irritation and localized edema.

Furthermore, several disorders are not amenable to self-help using injectables, although this is the most desirable method of treatment. For example, hypoglycemic crisis is preferably treated with intravenous, intramuscular or subcutaneous injections of glucagon or intravenous solutions of glucose solutions. Patients experiencing a hypoglycemic episode cannot easily treat themselves with injections, as their motor functions are impaired. However, treatment is crucial since prolonged hypoglycemia can lead to irreversible coma and even death. Generally, patients must resort to eating sugar candies, dextrose tablets or paste in order to raise the blood glucose concentration. This method is less than desirable since the substances must travel to the intestine for absorption and timing is crucial in such a crisis.

For these reasons, there is increased interest in new drug delivery systems such as delivery by inhalation or via administration by eye drops. U.S. Pat. Nos. 5,182,258, 5,278,142 and 5,283,236 describe such a system for the systemic delivery of drug via the ocular route. Since ease of use is one benefit of such a drug delivery system, it is necessary that the methods used to stabilize the drug and reconstitute the drug solution with high quantitative accuracy be as simple as possible. A preferred method of accomplishing this goal is to provide a dry form of the drug that can be precisely reconstituted to give a solution of known drug concentration. The requirements for such a dry form depend upon the method of use as well as the general requirements of providing a stable form of and precise quantity of the drug. For example, when preparing a single dose of drug for immediate use, the dry form of the drug must dissolve rapidly. However, if multiple doses are prepared for delivery over a relatively long period of time, quick dissolution may not be required for all doses since only the first dose may be administered immediately. Thus, methods for producing dry formulations of a drug that can be tailored to accommodate various delivery schemes would be of significant benefit.

A further practical difficulty arises because the process of dry mixing powders to make tablets and capsules cannot insure homogeneity. In addition, localized impurities may occur such that one tablet has a higher level of impurity than another. Moreover, even the dosage cannot be controlled with a high degree of precision without elaborate precautions due to problems associated with the blending of solid components in a formulation. Thus, tablets are best used for drugs with a wide therapeutic range.

Alternate methods of providing stabilized drugs have been reported. For example, U.S. Pat. Nos. 5,624,597 and 5,413,732 describe compositions useful for analytical chemical testing. The disclosures of these patents relate to the formation of lyophilized reagent spheres comprising reagents suitable for the analysis of blood samples. U.S. Pat. Nos. 3,721,725 and 3,932,943 relate to methods for producing lyophilized reagents comprising spraying the reagents into a moving bath of fluorocarbon refrigerants and lyophilizing the resultant frozen droplets. U.S. Pat. No 4,848,094 discloses methods for the generation of essentially spherical frozen droplets and improved methods for removing them from a cryogenic liquid. U.S. Pat. No. 4,655,047 describes methods for freezing drops of viscous liquids by dropping them from a small height into cryogenic material. U.S. Pat. Nos. 4,678,812 and 4,762,857 describe diagnostic tablets containing trehalose as an excipient and stabilizer. U.S. Pat. No. 5,275,016 describes an apparatus that can be used to prepare frozen drops using a cryogenic liquid. U.S. Pat. No. 4,982,577 describes an alternate apparatus for producing frozen beads.

While these patents discloses methods for preparing frozen drops of diagnostic reagents and the like, there are no known successful methods of preparing small, single-dose, precisely-measured dried or lyophilized solid spheres or beads comprising pharmaceuticals, particularly peptide or polypeptide drugs.

The present invention provides for the production of precisely measured solid doses of drugs, particularly peptide or polypeptide drugs for systemic disease, that are uniform in composition and weight and that can be adapted to control the rate of dissolution.

### SUMMARY OF THE INVENTION

The present invention is directed toward compositions for the delivery of precisely measured quantities of drugs in a stable, dry matrix and methods for preparing the same. The drugs in this stable, dry matrix are capable of dissolving in solution either immediately or over a longer, predetermined period of time so that drug dosage solutions can be prepared for immediate and/or future use. In a preferred embodiment, the stabilized, dry drug is prepared for delivery by ocular application and the dry matrix containing the drug is incorporated into a device optimized for ocular drug delivery as disclosed in co-pending U.S. patent application Attorney Docket No. 260332000900.

In accordance with the instant invention, a drug is dissolved in a solvent, such as water, along with fillers, such as polyethylene glycol, myo-inositol, polyvinylpyrrolidone, bovine serum albumin, dextrin, mannitol, trehalose, sodium carbonate, sodium bicarbonate, boric acid and its salts, dextrose, sodium acetate, sodium or potassium phosphates, polyvinyl alcohol-polyvinyl acetate copolymers, and the like. These fillers are used alone or in combination. Surfactants, such as Triton X-100®, sodium laurel sulfate, cetyl trimethyl ammonium chloride, and the like, may be added. Separate buffer components may also be added, if required. Preservatives may also be included in the formulation if the reconstituted solution is to be stored for any appreciable time. The drug and the filler(s) along with buffer components and surfactants, if desired, are dissolved to prepare an essentially homogeneous solution. The term homogeneous should not be interpreted to imply that colloids or micelles might not exist in the liquid phase. Colloids, micelles, and similar materials can exist as suspensions that behave mechanically as true solutions as is well known in the colloid chemistry art. The resulting solution may optionally be degassed prior to dispensing and is dispensed as precisely measured droplets. The droplet size is typically from about 1.5 to about 20 microliters. This process will typically produce dry beads ranging from about 1 to about 4 mm in diameter depending upon the solid content of the dispensed solution, its chemical composition, and the method used to dry the solid.

Lyophilization is a preferred method of drying beads that must dissolve rapidly. Droplets are produced by pumping the solution using a precise pump, usually of a direct displacement type, through an appropriate nozzle. The nozzle has an inside diameter ranging from 0.010 to 0.050 inches, preferably about 0.03 inches. The nozzle tip is typically tapered and has a wall thickness typically ranging from about 0.005 to about 0.020 inches depending upon the properties of the solution being dispensed. Pumps like an IVEK model AAA pump (N. Springfield, Vt.) are particularly suitable for this use. The solution is dispensed with a drop rate of from about 1 to about 3 drops per second. There is no lower limit to drop frequency and the upper limit is determined by the rate of solidification of the dispensed material.

The dispensed droplets fall into a liquid bath that causes the droplet to form into a solid sphere. The mechanism of sphere formation may be freezing, solvent incompatibility or chemical reaction or combinations thereof. In a preferred embodiment, spheres are formed by freezing which is accomplished by allowing the droplet to fall into a bath of liquid nitrogen. This method is used primarily to produce spheres that dissolve immediately since the freezing step is followed by a drying step, usually by lyophilization. Lyophilization produces spheres with low density. In other words, the solid mass has a large void volume.

In another embodiment, spheres are formed by solvent incompatibility. Solvent incompatibility occurs when the filler employed is slowly soluble in water but is highly soluble in a water miscible solvent such as ethanol, tetrahydrofuran, acetone, dimethylformamide, and the like. In this embodiment, the drug, fillers, buffers, and surfactants are dissolved in the solvent. Droplets of the resulting homogeneous solution are then dispensed into a large water bath. The water bath may be chilled and/or contain salts such as high concentrations of sodium chloride (brine solutions) to help solidify the beads. When the droplets fall into the water bath, the solvent mixes rapidly with the water and causes the drug to rapidly precipitate inside the sphere. In this embodiment, the percentage of filler in the solution must be high (typically >20% solids). The spheres are then filtered or otherwise removed from the water bath and air or oven dried. Lyophilization is unnecessary.

In another embodiment, a chemical reaction is employed to form the spheres. In this embodiment, the drug is dissolved in a solution of filler that can react chemically in a subsequent reaction. For example, a drug is dissolved in a concentrated solution of a high molecular weight polycarboxylic acid salt such as the sodium salt of styrene-maleic acid copolymer. This solution, which is viscous, is then dispensed into a water bath that includes a buffer at a pH well below the effective pKa of the styrene-maleic acid. Proton transfer occurs at an extremely rapid rate upon mixing of the solution with the water and causes the very water-soluble sodium salt of styrene-maleic acid into styrene-maleic acid, which is very slowly soluble. This reaction leads to the formation of a solid sphere of the acid that traps the drug. Once again, the resulting spheres must be filtered or otherwise removed from the water bath and air or oven dried. Lyophilization is necessary.

Suitable drugs for use within the instant invention include, but are not limited to, pharmaceuticals and peptide and polypeptide drugs such as glucagon, insulin, oxytocin, thyrotrophin releasing hormone (TRH), leucine-enkephalin-methionine-enkephalin, somatotropin, oxytocin, vasopressin, lypressin, alpha-neoendorphin, beta-neoendorphin, luteinizing hormone releasing hormone (LHRH), dynorphin A, dynorphin B, somatostatin, secretin, calcitonin, ACTH, growth hormone releasing hormone, concanavalin, ribonuclease, lysozyme, ribonuclease, beta-lipotropin, gamma-lipotropin, and the like.

The following examples illustrate methods for preparing drug delivery spheres for use in ocular drug delivery systems. These examples are provided for illustrative purposes only and are not intended to limit the scope of the present invention in any way. Other aspects, advantages and modifications within the scope of the invention will be apparent to those skilled in the art to which the invention pertains.

All the patents, patent applications, and references cited herein are hereby incorporated by reference in their entirety.

### EXAMPLE 1

Preparation of Rapidly Dissolving Spheres for Ocular Drug Delivery

Buffer Solution A was prepared by accurately weighing and dissolving 9.806 grams of CAPS buffer (3-cyclohexylamino-1-propane sulfonic acid) in 250 mL of deionized water. The pH was adjusted to 9.92.

Filler Solution B1 was prepared containing 5.51 grams of polyethylene glycol (MW 2000) plus 5.01 grams of polyethylene glycol (MW 3400) plus 6.006 grams of polyethylene glycol (MW 10,000) in 75 mL of Buffer Solution A.

Filler Solution B2 was prepared containing 17.504 of polyethylene glycol (MW 10,000) in 75 mL of Solution A.

Filler Solution B3 was prepared containing 15,008 grams of polyethylene glycol (MW 10,000) plus 5.5012 grams of polyethylene glycol (MW 3400) in 80 mL of Solution A.

Drug Solution C containing 15.6 mg of glucagon in 7.5 mL of deionized water was prepared.

Three dispense formulations were prepared.

Dispense Formulation F1 was prepared by adding 2.5 mL of Drug Solution C to 7.5 mL of Filler Solution B1. Dispense Formulation F1 contains 19.5 percent total solids.

Dispense Formulation F2 was prepared by adding 2.5 mL of Drug Solution C to 7.5 mL of Filler Solution B2. Dispense Formulation F2 contains 17.9% solids.

Dispense Formulation F3 was prepared by adding 2 mL of Drug Solution C to 8 ml of Filler Solution B3. Dispense Formulation F3 contains 23.5% solids.

These dispense formulations were separately dispensed using an IVEK Model AAA pump. The drops were adjusted to be 5 microliters in volume with a target weight of 5.3 mg. The drops were dispensed into a liquid nitrogen bath. The following results were obtained:

| Dispense Formulation | F1 | F2 | F3 |
|---|---|---|---|
| Dispense volume | 5 μL | 5 μL | 5 μL |
| Target Weight | 5.3 mg | 5 mg | 5 mg |
| Average weight | 5.323 mg | 5.299 mg | 5.284 mg |
| Standard Deviation | 0.039 mg | 0.153 mg | 0.027 mg |
| % CV | 0.7 | 2.9 | 0.5 |
| # samples | 5 | 5 | 5 |
| # beads produced | 1200 | 1200 | 1300 |

The resulting frozen spheres were then placed in a Vertis Freeze dryer model 12 EL (Gardener, N.Y.) and lyophilized overnight. The spheres had a residual moisture content below 5%. All spheres produced were white with a uniform appearance and a hard, smooth surface. When placed in water, each type of sphere dissolved completely in about 1 second.

### EXAMPLE 2

Preparation of Rapidly Dissolving Spheres for Ocular Drug Delivery with Higher Drug Content

Buffer Solution A is prepared by accurately weighing and dissolving 9.806 grams of CAPS buffer in 250 mL of deionized water. The pH is adjusted to 9.92.

Filler Solution B1 is prepared containing 5.51 grams of polyethylene glycol (MW 2000) plus 5.01 grams of polyethylene glycol (MW 3400) plus 6.006 grams of polyethylene glycol (MW 10,000) in 75 mL of Buffer Solution A.

Filler Solution B2 is prepared containing 17.504 of polyethylene glycol (MW 10.000) in 75 mL of Buffer Solution A.

Filler Solution B3 is prepared containing 15.008 grams of polyethylene glycol (MW 10,000) plus 5.5012 grams of polyethylene glycol (MW 3400) in 80 ml of Buffer Solution A.

Drug Solution C containing 232 mg of glucagon in 7.5 ml of deionized water is prepared.

Three dispense formulations are prepared.

Dispense Formulation F1 is prepared by adding 2.5 mL of Drug Solution C to 7.5 mL of Filler Solution B1. This solution contains 20.2 percent total solids.

Dispense Formulation F2 is prepared by adding 2.5 mL of Drug Solution C to 7.5 mL of Filler Solution B2. This solution contains 18.6% solids.

Dispense Formulation F3 is prepared by adding 2 mL of Drug Solution C to 8 mL of Filler Solution B3. This solution contains 24.1% solids.

These formulations are separately dispensed using an IVEK Model AAA pump. The drops are adjusted to be 5 microliters in volume with a target weight of 5.3 mg. The drops are dispensed into a liquid nitrogen bath.

The resulting frozen spheres are then placed in a Vertis Freeze dryer model 12 EL (Gardener, N.Y.) and lyophilized overnight. The spheres have a residual moisture content below 5%. All spheres produced are white with a uniform appearance and a hard, smooth surface. When placed in water, each type of sphere dissolves completely in about 1 second.

### EXAMPLE 3

Preparation of Rapidly Dissolving Spheres for Ocular Drug Delivery with Higher Drug Content and Alternate Solid Matrices

Buffer Solution A is prepared by accurately weighing and dissolving 9.806 grams of CAPS buffer in 250 mL of deionized water. The pH is adjusted to 9.92.

Filler Solution B1 is prepared containing 2 grams of dextran plus 6.0 grams of mannitol plus 1 grams of trehalose in 75 mL of Buffer Solution A.

Filler Solution B2 is prepared containing 17.5 grams of polyethylene glycol (MW 20,000) in 75 mL of Buffer Solution A.

Filler Solution B3 is prepared containing 1 gram of dextrin, 10 grams of mannitol, and 0.05 grams of Triton X100 in 80 mL of Buffer Solution A. Drug Solution C containing 232 mg of glucagon in 7.5 mL of deionized water is prepared.

Three dispense formulations are prepared.

Dispense Formulation F1 is prepared by adding 2.5 mL of Drug Solution C to 7.5 mL of Filler Solution B1. Dispense Formulation F1 contains 12.7 percent total solids.

Dispense Formulation F2 is prepared by adding 2.5 mL of Drug Solution C to 7.5 mL of Filler Solution B2. Dispense Formulation F2 contains 16.1% solids.

Dispense Formulation F3 is prepared by adding 2 mL of Drug Solution C to 8 mL of Filler Solution B3. Dispense Formulation F3 contains 22.8% solids.

These formulations are separately dispensed using an IVEK Model AAA pump. The drops are adjusted to be 5 microliters in volume with a target weight of 5.3 mg. The drops are dispensed into a liquid nitrogen bath.

The resulting frozen spheres are placed in a Vertis Freeze dryer model 12 EL (Gardener, N.Y.) and lyophilized overnight. The spheres have a residual moisture content below 5%. All spheres produced are white with a uniform appearance and a hard, smooth surface. When placed in water, each type of spheres dissolves completely in about 1 second.

### EXAMPLE 4

Preparation of Slowly Dissolving Spheres using Solvent Incompatibility

A solution of the drug sulfanilamide is prepared by dissolving 150 mg of the drug in 100 mL of tetrahydrofuran which also contains 20 grams of dissolved polyethylene-polyvinyl alcohol copolymer. This solution is dispensed using an EDF pump (East Providence Road Island) Model 1500XL or 2000XL with 5 microliter drop size into a 10 liter water bath held at 4° C. Upon hitting the water, the tetrahydrofuran and water mix and the water-insoluble polymer immediately precipitates out carrying the slightly water soluble drug with it: The spheres are immediately filtered and dried by blowing a stream of warm air over the spheres for 10 minutes.

For drugs that may be more water soluble, the water bath is presaturated with the drug before the organic solution is added. The spheres are then filtered, washed with a small volume of cold distilled water and dried as above.

### EXAMPLE 5

Preparation of Slowly Dissolving Beads using Chemical Reactions

A solution of the antidiuretic drug, hydroxydione sodium, containing 10 mg of drug per 100 mL of distilled water is prepared. This is a steroid-type drug with very low solubility in low pH solutions.

A solution of poly (ethylene-maleic anhydride) copolymer is prepared by treating 25 grams of the copolymer with boiling water until the anhydride is completely hydrolyzed. This process is accelerated by adding small amounts of concentrated NaOH to the hot mixture to neutralize the acid. At the end of the solution process, the resulting pH is about 10. After cooling, 90 mL of the copolymer solution is mixed with 10 mL of the drug solution. This solution is dispensed using an EDF pump (see above) with 5 microliter drop size into a 10 liter water bath containing 0.1M citrate buffer, pH 4. When the drop hits the solution in the bath, proton transfer occurs upon mixing and the vinyl-maleic acid copolymer precipitates from solution, carrying with it, the insoluble drug. The resulting spheres are filtered, washed with cold deionized or distilled water and air-dried. In use, the spheres are reconstituted with an alkaline solution such as a carbonate or borate buffer at pH 10. This causes the polymer to swell and allow the drug to dissolve in the alkaline medium.

What is claimed is:

1. A method for preparing pharmaceutical spheres comprising pharmaceuticals for administration to a subject in need thereof, the method comprising the steps of:
(a) dissolving a pharmaceutical in a solvent to form a homogeneous solution;
(b) adding at least one filler to the homogeneous solution of step a;
(c) optionally adding a buffer component to the solution produced in step b;
(d) optionally adding a surfactant to the solution produced in step b;
(e) dispensing precisely measured drops of the resulting solution into a liquid bath to produce solid spheres; and
(f) separating the spheres formed in step e from the liquid bath.

2. The method of claim 1 wherein the liquid bath comprises a cryogenic liquid, whereby the drops are frozen.

3. The method of claim 2 further comprising the step of lyophilizing the frozen drops, thereby forming dry pharmaceutical spheres.

4. The method of claim 1 wherein the liquid bath comprises a solvent that is miscible with the solvent of step a and further wherein the pharmaceutical and filler are only slightly soluble.

5. The method of claim 4, further comprising the step of air drying the separated spheres.

6. The method of claim 1 wherein the liquid bath comprises at least one compound that will precipitate the filler.

7. The method of claim 6, further comprising air drying the separated spheres.

8. The method of claim 1 wherein the pharmaceutical is a peptide.

9. The method of claim 1 wherein the pharmaceutical is a polypeptide.

10. The method of claim 9 wherein the polypeptide is selected from the group consisting of glucagon, insulin, oxytocin, thyrotrophin releasing hormone (TRH), leucine-enkephalin, methionine-enkephalin, somatotropin, oxytocin, vasopressin, lypressin, alpha-neoendorphin, beta-neoendorphin, luteining hormone releasing hormone (LHRH), dynorphin A, dynorphin B, somatostatin, secretin, calcitonin, ACTH, growth hormone releasing hormone, concanavalin, ribonuclease, lysozyme, ribonuclease, beta-lipotropin and gamma-lipotropin.

11. The method of claim 9 wherein the pharmaceutical is glucagon.

12. The method of claim 9 wherein the pharmaceutical is insulin.

13. The method of claim 1 wherein the filler material is selected from the group consisting of polyethylene glycol, myo-inositol, polyvinylpyrrolidone, bovine serum albumin, dextrin, mannitol, trehalose, sodium carbonate, sodium bicarbonate, boric acid and its salts, dextrose, sodium acetate, sodium or potassium phosphates and polyvinyl alcohol-polyvinyl acetate copolymers.

14. The method of claim 1 wherein the surfactant is selected from the group consisting of Triton X-100®, sodium laurel sulfate and cetyl trimethyl ammonium chloride.

15. The method of claim 1 further comprising the step of adding a preservative to the resulting solution.

16. A composition comprising a solid sphere comprising a pharmaceutical and at least one filler material made in accordance with the method of claim 1.

17. A composition comprising a dry, solid sphere comprising a pharmaceutical in a precisely measured amount and at least one filler material in an amount sufficient to facilitate the formation of a matrix capable of conducting a solution into the sphere.

18. The composition of claim 17 further comprising a surfactant.

19. The composition of claim 17 further comprising a buffer component.

20. The composition of claim 17, wherein the pharmaceutical is a peptide.

21. The composition of claim 17, wherein the pharmaceutical is a polypeptide.

22. The composition of claim 21 wherein the polypeptide is selected from the group consisting of glucagon, insulin, oxytocin, thyrotrophin releasing hormone (TRH), leucine-enkephalin, methionine-enkephalin, somatotropin, oxytocin, vasopressin, lypressin, alpha-neoendorphin, beta-neoendorphin, luteininig hormone releasing hormone (LHRH), dynorphin A, dynorphin B, somatostatin, secretin, calcitonin, ACTH, growth hormone releasing hormone, concanavalin, ribonuclease, lysozyme, ribonuclease, beta-lipotropin and gamma-lipotropin.

23. The composition of claim 21 wherein the polypeptide is glucagon.

24. The composition of claim 21 wherein the polypeptide is insulin.

25. The composition of claim 17 wherein the amount of the pharmaceutical is sufficient for a single dose administered immediately upon dissolution of the sphere in a solution.

26. The composition of claim 17 wherein the amount of the pharmaceutical is sufficient for sustained release administration over a predetermined period of time.
